(19) United States
(12) Patent Application Publication
(10) Pub. No.: US 2021/0371785 A1
(43) Pub. Date: Dec. 2, 2021

(54) Disposable Cell for In Situ Monitoring Probe

(71) Applicant: Nirrin Technologies, Inc., Billerica, MA (US)

(72) Inventors: Bryan A. Hassell, Cambridge, MA (US); David P. Marchessault, Hopkinton, MA (US)

(21) Appl. No.: 17/337,055

(22) Filed: Jun. 2, 2021

Related U.S. Application Data
(60) Provisional application No. 63/033,521, filed on Jun. 2, 2020.

(51) Int. Cl. C12M 1/00; C12M 1/34; C12M 1/36; C12M 3/04

(52) U.S. Cl. CPC C12M 23/14; C12M 23/28; C12M 27/10; C12M 41/48; C12M 41/36

(57) ABSTRACT

An in situ probe, comprising reusable and disposable components, can be employed to measure cell viability in a rocking bioreactor.

### RELATED APPLICATIONS

This application claims the benefit under 35 USC 119(e) of U.S. Provisional Application No. 63/033,521, filed on Jun. 2, 2020, which is incorporated herein by reference in its entirety.

### BACKGROUND OF THE INVENTION

Many processes in the chemical, biochemical, pharmaceutical, food, beverage and in other industries require some type of monitoring.

Sensors have been developed and are available to measure pH, dissolved oxygen (DO), temperature or pressure in-situ and in real-time. Common techniques for detecting chemical constituents include high performance liquid chromatography (HPLC), gas chromatography-mass spectroscopy (GCMS), or enzyme- and reagent-based electrochemical methods.

While considered accurate, many existing approaches are conducted off-line, tend to be destructive with respect to the sample, often require expensive consumables and/or take a long time to complete. In many cases, the equipment needed to perform these analyses is expensive, involves complex calibrations, and trained operators. Procedures may be time- and labor-intensive, often mitigated by decreasing the sampling frequency of a given process, thus reducing the data points. Often, samples are run in batches, after the process has been completed, yielding little or no feedback for adjusting conditions on an ongoing basis. Drawbacks such as these can persist even with automated sampling operations.

Various optical spectroscopy approaches are available to assess components, also referred to as analytes, in a sample. Among these, probably the most common is absorption spectroscopy. Incident light excites electrons of the analyte from a low energy ground state into a high energy, excited state, and the energy can be absorbed by both non-bonding n-electrons and π-electrons within a molecular orbital. Absorption spectroscopy can be performed in the ultraviolet, visible, and/or infrared region, with analytes of varying material phases and composition being interrogated by specific wavelengths or wavelength bands of light. The resulting transmitted light is then used to resolve the absorbed spectra, to determine the analyte's or sample's composition, temperature, pH and/or other intrinsic properties for applications ranging from medical diagnostics, pharmaceutical development, food and beverage quality control, to list a few.

Another option is Raman spectroscopy, which works by the detection of inelastic scattering of typically monochromatic light from a laser.

### SUMMARY OF THE INVENTION

Robust, hands-free, non-destructive techniques for identifying and/or quantifying constituents in a given process in real time are highly desirable. Typically, the process is conducted in a vessel, e.g., a bioreactor. The contents of the bioreactor can change as the process unfolds and data collected at various stages can be used to monitor, adjust and/or control process parameters.

Recently, techniques for monitoring bioreactors have been described. For instance, see U.S. Pat. Pub. No. US 2021/0088433 with the title In Situ Probe, filed on Sep. 23, 2020. U.S. Pat. No. 9,404,072 B2, issued to Koerperick et al. on Aug. 2, 2016 describes flange-based arrangements for optical sampling in flexible bioreactors.

Nevertheless, a need continues to exist for methods and equipment designed to monitor processes conducted in bioreactors, such as, for example, in flexible bioreactors, also referred to herein as “bag bioreactors” or “rocking bioreactors”. Monitoring devices that can be integrated in the bioreactor design are particularly desirable, as are probes that can include disposable and possibly reusable components. Also of interest are techniques and equipment for measuring cell viability.

Generally, bags for reactors are single use, flexible bags made of polymeric materials. The bags may be used with a rocking system or an internal impeller can be used. They can be configured in a variety of sizes and are often pre-sterilized. Easy to handle, often inexpensive, these bags offer time and labor advantages. Nevertheless, these flexible bags present some challenges, when wishing to analyze process ingredients, for example.

Aspects of the invention feature approaches that employ an in-situ probe for monitoring a process conducted in a flexible bag of a bioreactor or other reservoir. That said, it can be used on other types of bioreactors or other systems where optical spectroscopic analysis of fluids is desired.

In specific embodiments the probe includes a disposable component configured to possibly receive a reusable component. The disposable component can be fused inside the flexible bag of a rocking bioreactor and will often include a detection assembly. The reusable component can be designed to fit or slide into the disposable component, make the electrical connection and provide the interrogation signal. In one implementation, the reusable component includes a vertical cavity surface emitting laser (VCSEL). In addition, the reusable component may be provided with an input/output (I/O) connector, a pin connector to the detector and a mounted lens assembly for collimating the VCSEL light. In still other examples, the whole system is disposable. This is especially the case when low-cost VCSELs are employed.

The probe described herein can be part of a system that includes a rocking bioreactor or other reservoir and a controller for conducting the analysis of the fluids in the reservoir. With the probe in place, the contents of the reactor can be analyzed spectroscopically, using, for instance near infrared absorption spectrometry. In one application, the techniques and equipment described herein measure cell viability.

Whereas many existing approaches rely on removing and/or circulating cells in loops external to the process vessel, typically through a pumping system, an in-situ probe can reduce, minimize and often eliminate the exposure of the bag contents to external conditions. In addition, cells are prevented from being drawn into a pumping system, where they could become damaged. The low sheer rocking motion often used with flexible reactors, as well as the absence of stirrers, impellers and the like, are other factors that contribute to protecting cells from damage, while also minimizing contamination.

Embodiments described herein reduce manual intervention, increasing reproducibility from one run to the next, streamlining the process, and diminishing the potential for errors. Detachable parts that can be assembled and disassembled as needed offer flexibility and convenience. In many cases, the analysis process according to embodiments disclosed herein is simplified and/or accelerated.

Advantageously, reactors come in a wide range of sizes, to accommodate many applications and needs. Typically pre-sterilized and designed for single use, bag reactors reduce the need for time-consuming vessel clean-up steps. Streamlining the equipment and protocols associated with a particular bioreactor process are enhanced when using a probe having a disposable component that is an integral part of a single use flexible bag.

Embodiments described herein present a compact design of a probe that can be at least partially confined within the bioreactor. For many implementations, the in situ probe is located at the bottom of the flexible bag, a configuration more likely to maintain the sample detection area of the probe submerged, regardless of the rocking motion.

The probe design can be optimized for good heat transfer between the heated plate and the bottom of the bag, e.g., by reducing bunching or the footprint occupied by fused ports.

The tunable vertical cavity surface emitting lasers (VCSELs) incorporated in some of the reusable modules described herein can supply superior spectral properties relative to other IR sources such as high-power LED and edge-emitting laser diodes (EELD). For example, NIR VCSEL often present a narrow laser spectrum, a stable wavelength and can be less wavelength dependent on temperature effects. In addition, the nature of the VCSELs make them lower cost to manufacture in a package.

Specific embodiments of the invention can be adapted to monitoring cell viabilities and rely on an integrated aberrated phase front measurement system in which a Fabry Perot contrast measurement is directly proportional to the aberrations (i.e. the number of live cells and/or the refractive index of those cells that are causing refractive index changes across the beam's phase front).

In general, according to one aspect, the invention features an in situ probe, comprising a disposable component that is attached to a bioreactor bag.

In embodiments, the disposable component is fused to the bioreactor bag. An annular flange can be used for this purpose.

A bottom piece can also be employed that mates with the disposable component. This piece may or may not be disposable.

The probe will often include a radiation source and a detector for detecting radiation generated by the radiation source.

In some cases, the radiation source is contained in a bottom piece that mates with the disposable component. The detector is then contained in a disposable component.

In other cases, the radiation source is contained in the disposable component. Then, the detector might be contained in a bottom piece that mates with the disposable component.

In most cases, a controller is used for controlling the probe to resolve the spectral response of contents of the bioreactor bag.

In general, according to one aspect, the invention features a rocking bioreactor comprising: a bioreactor bag and an in situ probe attached to the bioreactor bag. The invention also extends to a process for monitoring the rocking bioreactor.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIGS. 1A and 1B are views illustrating the components and principles of operation of a rocking bioreactor;

FIGS. 2A, 2B and 2C are side elevation, top perspective, side exploded views of an in-situ probe according to the present invention;

FIG. 3A is a cross-sectional exploded view of a disposable component and reusable component of the in situ probe, according to embodiments of the invention;

FIG. 3B is a cross-sectional view of the assembled in situ probe, according to embodiments of the invention;

FIG. 4 is an perspective view of a bottom module including a VCSEL that can be employed in an in situ probe such as that in FIGS. 2A and 3B;

FIG. 5A is a side view of another embodiment of an in situ probe that can be used to practice aspects of the invention;

FIG. 5B is an perspective view of the embodiment of FIG. 5A;

FIG. 6 is a diagram of a rocking bioreactor including an in situ probed according to embodiments of the invention; and

FIG. 7 is a diagram illustrating principles employed to conduct cell viability measurements using an in situ probe such as that in FIGS. 2A and 3C.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The invention generally relates to an arrangement and/or method for monitoring an ongoing process, particularly a biological process. In many of its aspects, the invention relates to approaches for analyzing the contents of a bioreactor as a function of time, using, for example, an in-situ probe. Cells and/or substances can be identified and often quantified using a suitable technique. Furthermore, cells and/or other constituents can be detected, at various time intervals, and observed, e.g., in real time, as their concentration may fluctuate or as they are generated or consumed. Examples of processes that can be monitored include cell growth protocols, fermentations, and so forth. Specific implementations of this invention relate to cell viability measurements.

Many of the techniques described herein are practiced with a flexible bioreactor, also known as a “rocking” or “bag” bioreactor. Some examples of commercially available rocking bioreactors include: BIOSTAT® RM TX & Flexsafe® RM TX Bags from Sartorius Stedim Biotech; ReadyToProcess WAVE™ 25 Bioreactor System from GE Healthcare; HyPerforma Rocker Bioreactor from ThermoFisher Scientific; and others. Rocking bioreactors can accommodate various process volumes (from microliters to many liters) and support various operations including, for instance, media preparation, cell cultivation, process development or optimization, microbial processes, etc.

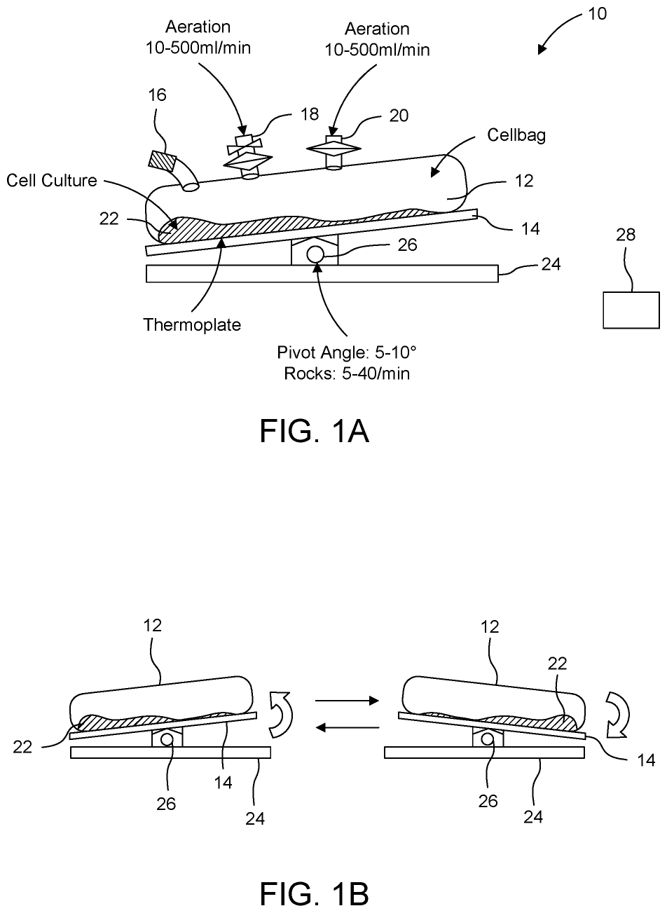

In a typical rocking reactor, such as rocking bioreactor 10 in FIGS. 1A and 1, the process is conducted in a flexible bag 12 disposed onto rocking plate 14, which can be heated. Bag 12 can be provided with access ports (see ports 16, 18 and 20 in FIG. 1A) that are typically fused to the bag wall. Ports to a rocking bioreactor can be used to load ingredients, aeration (at a rate of from about 10 to about 500 ml/minute, for example), end-of-run drainage, and so forth. In some designs, multiple (two or more) bags can be rocked on a single rocking platform as illustrated in the rocking bioreactors shown at https://www.gelifesciences.com/en/us/shop/cell-culture-and-fermentation/rocking-bioreactors/systems/readytoprocess-wave-25-rocker-p-05542 and at https://www.sartorius.com/us-en/products/fermentation-bioreactors/single-use-bioreactors/biostat-rm-flexsafe-rm.

In many implementations, bag 12 is a flexible, single use (disposable) container that can be pre-sterilized. It can be provided in a collapsed state and inflated in place, using suitable instrumentation. Typically, the bags are made of USP (U.S. Pharmacopeia) Class VI plastics (resins certified for medical applications). Many designs involve a multi-layer (or “multi-film”) arrangement.

In one example, an outer layer, offering mechanical stability, is made from a semi-rigid thermoset such as polyethylene terephthalate or low density polyethylene (LDPE). A second layer, often made of polyvinyl acetate (PVA) or polyvinyl chloride (PVC), controls gas transfer. The interior or “contact” layer is made of PVA or polypropylene (PP).

In another example, the bag is made from multilayered USP Class VI plastics having a contact surface which is an ethylene-vinyl acetate (EVA)/LDPE copolymer and an outer layer designed to provide flexibility, strength, and extremely low gas permeability.

Generally, rocking bioreactors do not employ stirring devices inside the bag. Rather, mixing and gas transfer are promoted by waves established in fluid 22, e.g., a cell culture, contained in bag 12. The waves are induced by a rocking motion (as illustrated in FIG. 1), often a gentle, low sheer rocking motion, provided, for instance, by a motorized base 24 which includes pivot 26. In many configurations, the motorized base is adjustable with respect to the rocking speed (e.g., within the range of from about 5 to about 40 rocks per minute) and angle (e.g., within the range of from about 5 to about 10 degrees). For cell cultures, the rocking motion also can prevent cells from settling.

Culture conditions such as dissolved oxygen, temperature, and pH can be monitored. Automated systems, using controller 28, for example, streamline operations and minimize the need for manual interventions.

To monitor the presence and/or concentration of ingredients on an ongoing basis, as the process unfolds, a rocking bioreactor such as the one shown in FIGS. 1A-1B is provided with an in-situ analysis probe, e.g., for spectrometric measurements. In many embodiments, the techniques employed to detect cells or other process ingredients such as, for example, culture media, nutrients, metabolites, enzymes, hormones, cytokines and so forth utilize near infrared (NIR) spectroscopy.

Probing molecular overtone and combination vibrations, NIR spectroscopy covers the region of from 780 nanometer (nm) to 2500 nm of the electromagnetic spectrum. An overview of NIR spectroscopy can be found, for example, in an article by A.M.C. Davies in “An Introduction to Near Infrared (NIR) Spectroscopy”, http://www.impublications.com/contest/introduction-near-infrared-nir-spectroscopy. See also, Cervera, A. E., Petersen, N., Lantz, A. E., Larsen, A. & Gernaey, K. V. Application of near-infrared spectroscopy for monitoring and control of cell culture and fermentation, Biotechnol. Prog. 25, 1561-1581 (2009); and Roggo Y, et al., “A review of near infrared spectroscopy and chemometrics in pharmaceutical technologies”, Journal of Pharmaceutical and Biomedical Analysis, Volume 44, Issue 3, 2007.

That said, more generally the analysis can be in one or more of the following electromagnetic spectral regions: millimeter, microwave, terahertz, infrared (including near-, mid- and/or far-infrared), visible, ultraviolet (UV), x-rays and/or gamma rays. Further, the spectroscopy system can measure different characteristics, such as absorption spectra, emission (including blackbody or fluorescence) spectra, elastic scattering and reflection spectra, impedance (e.g., index of refraction) spectra, and/or inelastic scattering (e.g., Raman and Compton scattering) spectra, of analytes in the bioreactor.

Illustrative embodiments described herein rely on spectroscopy in the ultraviolet, visible regions, and/or the infrared region, e.g., extending from 700 nanometers (nm) to 1 millimeter (mm) in wavelength and specifically including the near infrared (0.78-2.5 microns (m), NIR), mid-wavelength infrared (3-8 μm, MWIR), long-wavelength infrared (8-15 μm, LWIR), and the far infrared (15-1000 μm, FIR) of the spectrum.

According to embodiments described herein, measurements are taken within (inside) the bag of the bioreactor, typically without a need to withdraw a sample from the reactor and direct it to a sample cell or to an external (ex-situ) arrangement for taking a reading. Thus, in many of its aspects, the invention relates to an in situ probe. The probe includes: a disposable component (also referred to herein as “part”, “piece” or “module”); and a disposable or reusable component (also referred to herein as “part”, “piece” or “module”). When fused or otherwise permanently bonded to a single use flexible bag, the disposable component can be discarded along with the bag. The in situ probe is assembled or disassembled by attaching or detaching the reusable component into or out of the disposable component.

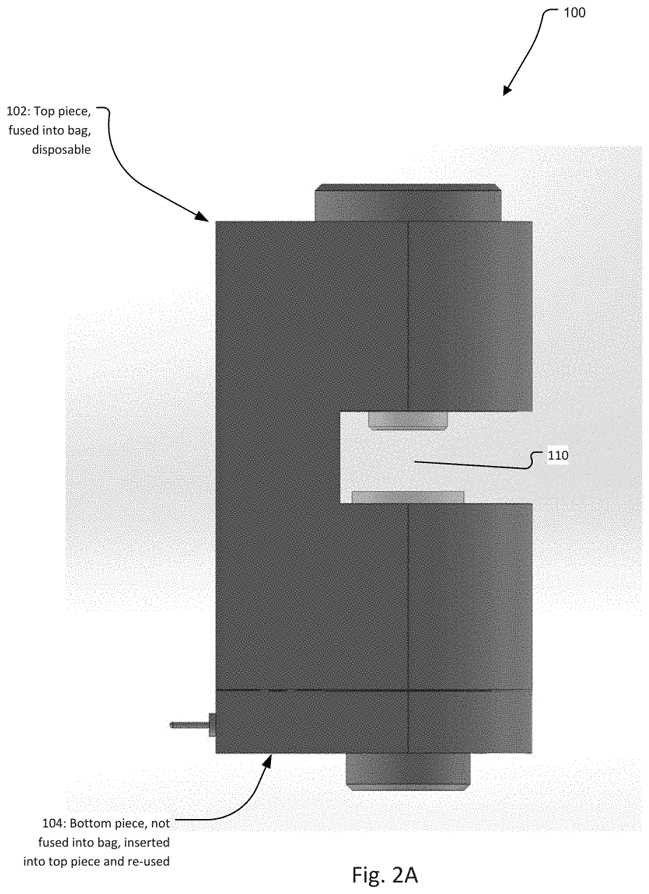

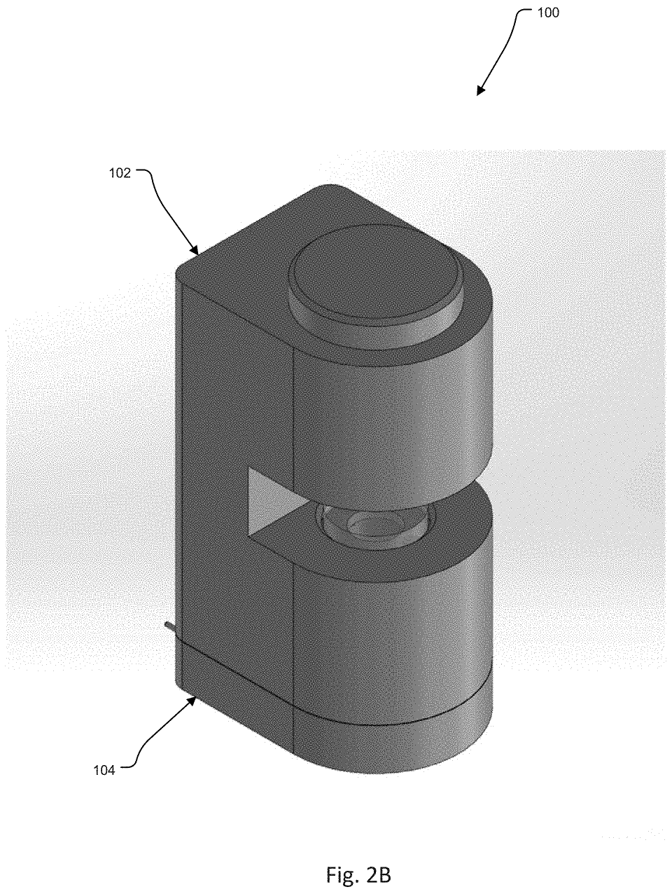

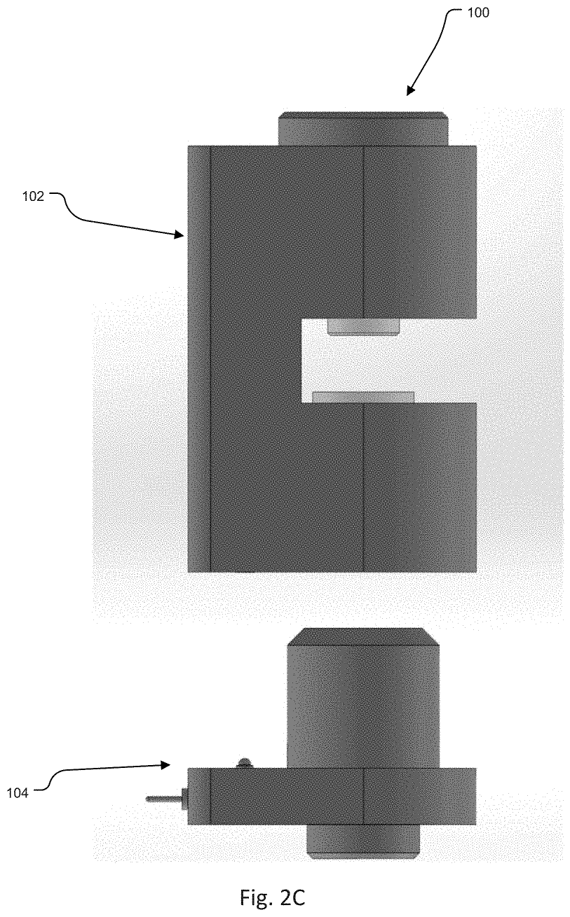

Shown in FIGS. 2A, 2B and 2C is in situ probe 100 which includes a disposable component, namely top piece 102, and a disposable or reusable module, namely bottom piece 104. Top piece 102 can be fused to the interior of a flexible bag, employing a suitable technique. Not designed to be fused to the interior of the bag, bottom piece 104 can be inserted into the top piece for assembly of the in situ probe. The in situ probe is disassembled by removing the reusable bottom module 104.

As shown, the in situ probe 100 is generally horse-shoe shaped with a sample detection volumetric region 110 between the two ends. Electromagnetic radiation is projected into this gap such as millimeter, microwave, terahertz, infrared (including near-, mid- and/or far- infrared), visible, ultraviolet (UV), x-ray and/or gamma ray radiation. This radiation interacts with the contents of a bioreactor that flows into the sample gap S. Then the absorption spectra, emission (including blackbody or fluorescence) spectra, elastic scattering and reflection spectra, impedance (e.g., index of refraction) spectra, and/or inelastic scattering (e.g., Raman and Compton scattering) spectra, is detected.

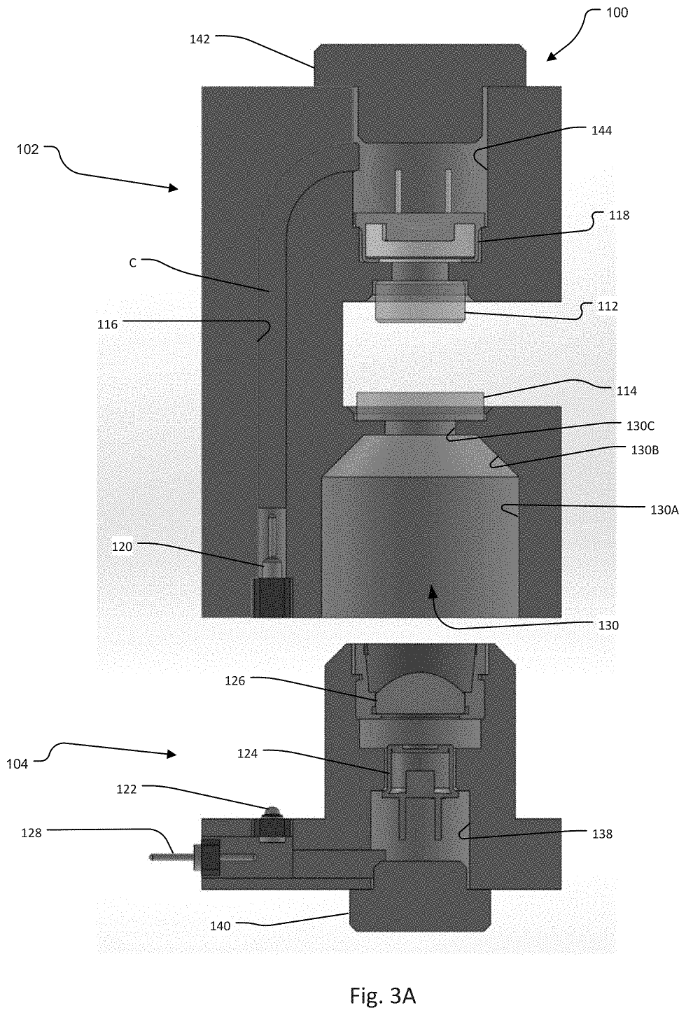

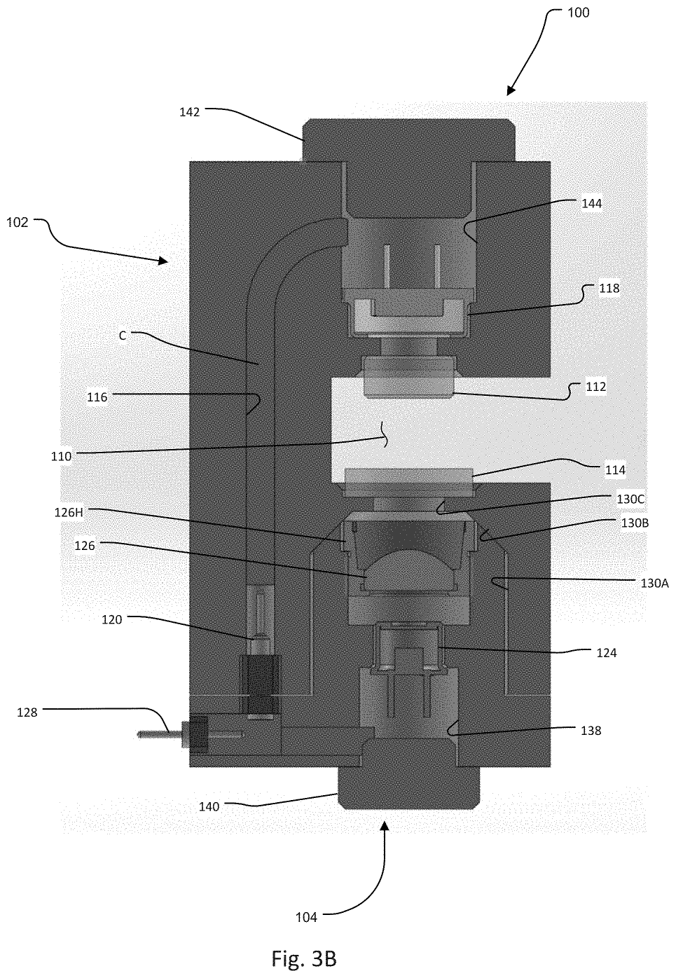

FIG. 3A is a cross-sectional view of the top and bottom pieces 102 and 104, respectively, while FIG. 3B is a cross-sectional view of the assembled in situ probe 100. Often the top piece 102 is attached to the interior of a disposable, flexible bag, resulting in an integrated design in which the disposable component can be discarded along with the bag.

A sample detection volumetric region 110 is defined by windows 112 (the detector window) and 114 (emitter window), which seal the interior parts of the in-situ probe and isolate them from the contents of the reactor. As these elements are in contact with the fluid inside the bioreactor, their spacing can also be the pathlength of the laser light. Specifically, the fluid-tight seal of the window 114 to an optical port 130C ensures that the bioreactor remains sealed and fluid does not leak out though the probe.

Top piece 102 is provided with a conduit 116 for detector cabling C to detector 118, such as a silicon photodetector, for instance. Pin connector 120 for the detector, typically a POGO pin connector, is designed to connect to pin connector 122 of the insertable module, namely the bottom piece 104 in FIG. 3A. The shape of the conduit 116 supporting cabling C will depend on factors such as the size of various components, manufacturing considerations, and others. For example, the curved bend toward the photodetector (region 146 in FIGS. 3A and 3B) is an entirely optional element; two straight cable channel intersecting conduits, leading from connector 120 to photodetector 118, are provided in some embodiments to aid in manufacturability.

As shown in FIG. 3B, this bottom piece includes a radiation generator such as a tunable laser or specifically a tunable VCSEL 124, lens 126 for collimating the VCSEL light, and input/output (I/O) pins 128. Lens 126 can be part of a mounted lens assembly (typically disposed beneath window 114), in which lens 126 is supported in lens holder 126H.

As shown in FIGS. 3A and 3B, the VCSEL 124 and the lens assembly (which includes lens 126 held in lens holder 126H) are mounted in a cavity 138 formed into the bottom piece 104. This cavity is sealed with a bottom cap 140.

At the same time, photodetector 118 is mounted in a cavity 144 formed into the top piece 102. This cavity is sealed with a top cap 142.

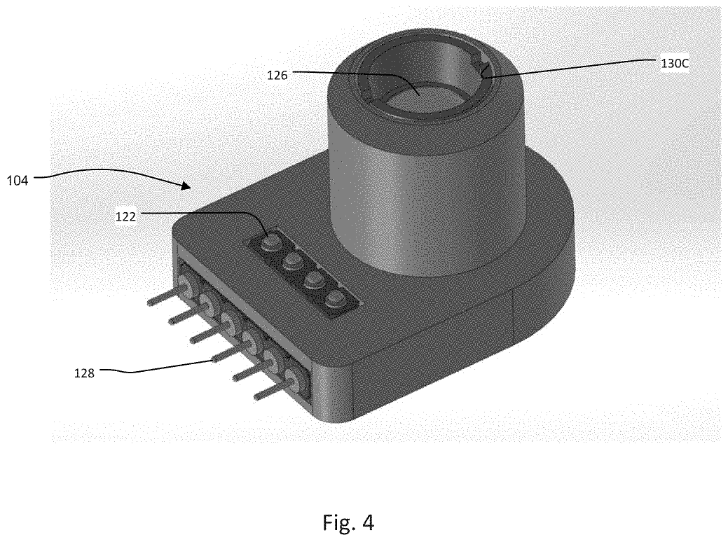

Various types of laser diodes or VCSELs are known or are being developed, covering continuous wavelength (CW), quasi CW or pulsed applications. Common available center wavelengths are 830, 976, 1064 nm. The tunable VCSEL generates a narrow band emission that is swept through a scan band around the center wavelengths. Often this scan band is greater than 10 nm and preferably greater than 30 nm. Other wavelengths within the range between 630 and 1064 nm can be supplied in some cases. Employing VCSELs in conjunction with a low-cost silicon photodetector may push the desirable wavelength to the long end of the spectrum, in the range between 800 and 900 nm, for example. An isometric view of a suitable VCSEL 124 is shown in FIG. 4.

To assemble the in situ probe 100, bottom piece 104 is fitted or slid into an opening 130, a space formed within the top piece 102 and shaped to receive the bottom piece. In the illustrated example, the opening 130 is cylindrical 130A with a fusto conical upper profile and ending in a short cylindrical section 130C functioning as an optical port ending at the lower surface of the laser window 114. The electrical connection is established by bringing into contact connectors 120 and 122. A view of the assembled probe 100 is shown in FIG. 3B. The in situ probe can be disassembled by breaking the contact between connectors 120 and 122 and pulling out bottom piece 104 from space 130. In this way, connecting to input/output (I/O) pins 128 enables the driving and powering of the VCSEL 124 and the monitoring of the response of the photodetector through a single electrical connection.

In some embodiments, both the bottom piece 104 and the top piece 102 are disposable. In addition, the locations of the laser 124 and the photodetector 118 can be reversed. That is, the laser is installed in the top piece, which is permanently affixed to the flexible bag 402 and the detector is located in the bottom piece 104 that can be disassembled from the top piece.

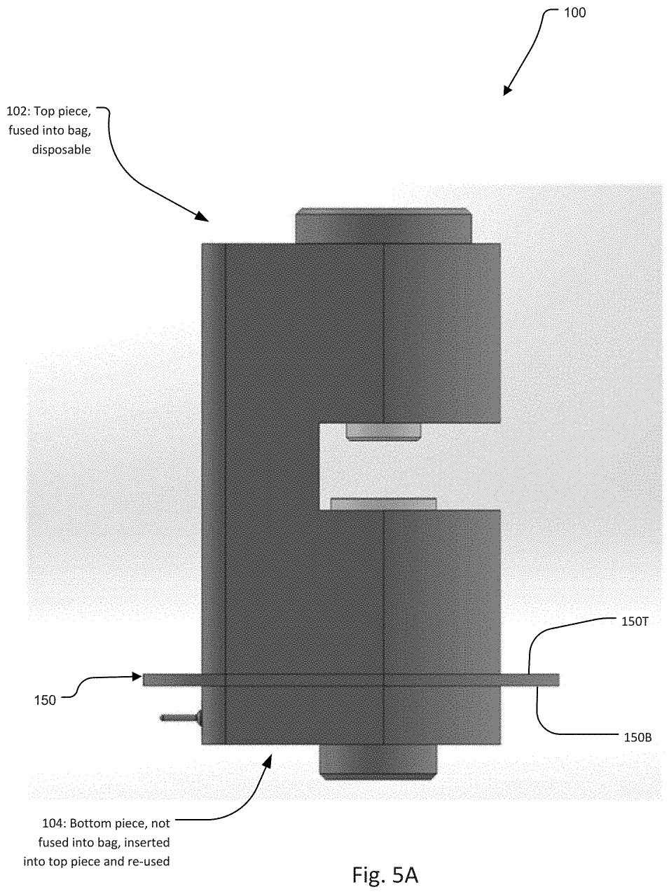

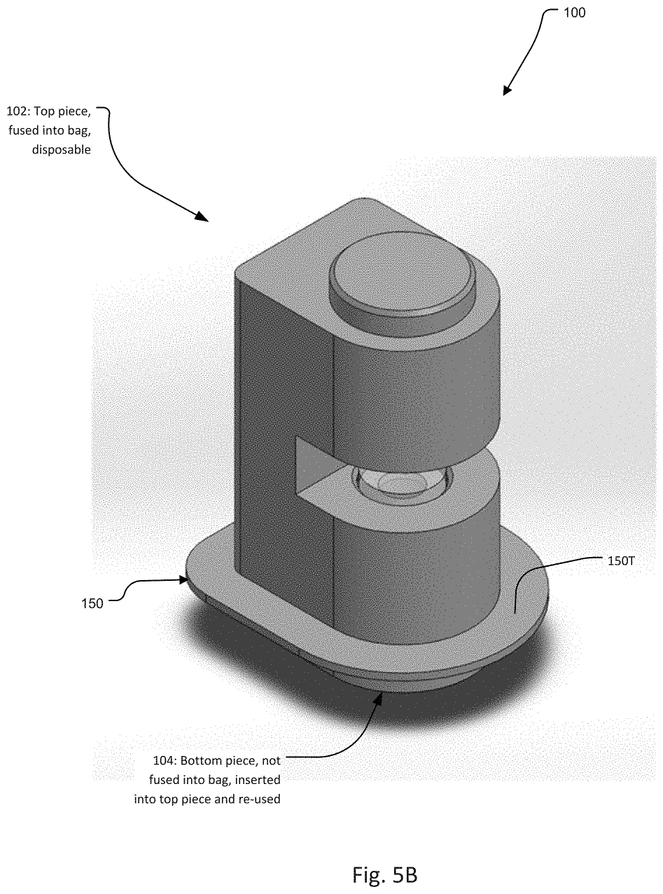

To facilitate attachment to the bag reactor, top piece 102 can be provided with an annular flange. Shown in FIGS. 5A and 5B, for example, is in situ probe 100 (in its assembled configuration). Probe 100 includes the top piece 102 and bottom piece 104, essentially as described above, and flange 150, extending outwardly at the bottom of top piece 102 forming an annular ring for creating a fluid tight seal to the bag. The flange is usually integral with the top piece 102.

Various arrangements can be employed to attach one of the flange surfaces (top surface 150T and bottom surface 150B in FIGS. 5A and 5B) to a bag for a rocking bioreactor. Selecting a suitable approach can depend on ease of manufacture, integration with an existing production process, or other considerations. In one example, flange 150 is internal to the bag, with bottom surface 150B being fused to the inner lining of the bag. In another example, the flange is at the exterior of the bag, with top surface 150T being fused to the outer face of the bag. Further designs can include a dual flange arrangement for a sandwich configuration in which the bag wall is clasped in between upper and lower flanges. For instance, a top surface of the lower flange can attach to the exterior of the bag and the bottom surface of the upper flange can attach to an interior lining in the bag.

Further aspects of the invention relate to a system in which the in situ probe is employed to monitor a rocking bioreactor.

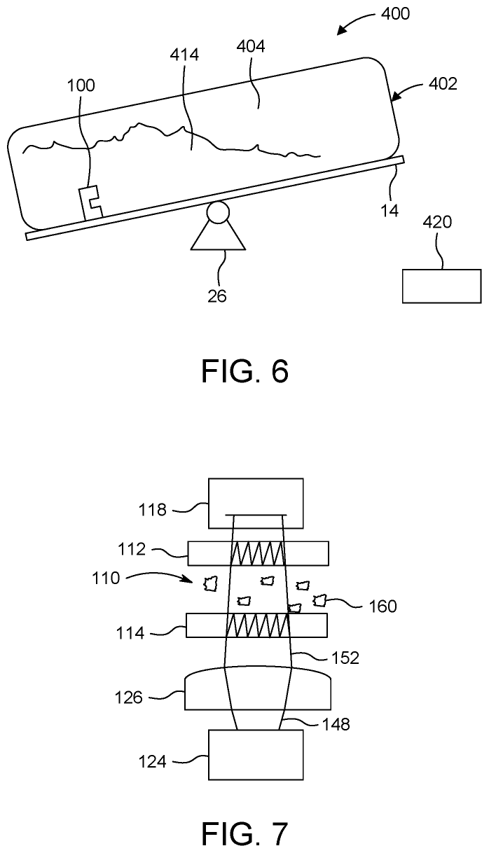

As shown in FIG. 6, system 400 includes flexible bag 402, in its inflated configuration. The bag is supported by plate 14, typically heated and (gently) rocked, essentially as described with reference to FIGS. 1A and 1B. The contents 414 present in the interior 404 of bag 402 can be monitored by in situ probe 100, located at the bottom of the bag.

The in-situ probe and system described herein can be monitored and/or controlled automatically, using controller 420, for example. The controller can be a single board computer performing functions that include but are not limited to monitoring the response of the photodetector during the analysis, setting scanning parameters, e.g., according to a suitable protocol, and so forth.

In typically operation, the controller 420 monitors the response of the photodetector 118 via the electrical connections 120, 122, 128. Thus, the controller 420 can resolve the absorption spectra of the sample by driving the spectral scanning of the tunable laser 124 over its scan band relative to the time-response of the photodetector 118. Generally, the tunable laser or tunable laser system sweeps its narrow band emission over some region of the electromagnetic spectrum such as the NIR and/or short-wave infrared (SWIR) regions, or portions thereof.

One application for the techniques and equipment disclosed herein relates to monitoring cell viability. Examples of cells include but are not limited to mammalian, bacterial, fungus, and many others.

As described by Hassell et al. in U.S. Patent Pub. No. US 2021/0140881, with the title Fabry Perot Interferometry for Measuring Cell Viability, filed on Nov. 11, 2020 and incorporated herein by this reference in its entirety, the life cycle of cells is generally marked by various changes. For instance, before autolysis (i.e., the destruction of cells or tissues by their own enzymes, such as enzymes released by lysosomes) cells will typically stain a deep red. As autolysis progresses, the staining becomes gradually fainter, probably due to losses in stainable material, and the cells appear disorganized. In addition, cells undergoing autolysis have an index of refraction that approaches the index of refraction of the aqueous cell culture, possible due to a loss in cell density and/or other mechanisms. In contrast, live cells have an index of refraction that is different from that of the aqueous culture medium, resulting in a turbid environment.

If directed through the cell culture medium, a phase front of light becomes distorted by the turbidity caused by live (viable) cells. Moreover, the distortion increases as the viable cell density increases. In turn, distorted wavefronts of the beam result in a reduction in interference contrast for an etalon forming a window into the cell culture medium.

Embodiments of the in situ probe described herein can be used in an integrated aberrated phase front measurement system in which a Fabry Perot contrast measurement is directly proportional to the aberrations (i.e. the number of live cells and/or the refractive index of those cells that are causing refractive index changes across the beam's phase front).

For such applications, one or both windows 114 and 112 in FIGS. 3A and 3B can be configured to form an etalon for generating a high frequency signal that can become superimposed onto a single beam transmission signal through the wavelength scan, obtained, for instance, in the near infrared (NIR) region available with the tunable VCSEL employed. As described in U.S. Provisional Patent Application No. 62/933,583, a mirror etalon can be established in optical elements that are devoid of their typical antireflective (AR) coating (often a thin dielectric film).

Shown in FIG. 7 is an arrangement in which light 148, generated by VCSEL 124 propagates through the lens 126. Provided with an AR coating, lens 126 allows the light to pass though and focus (without some of it being reflected) generating a spherical wave-front 152, which passes through laser window 114, detection area 110 and detector window 112.

Applying principles described in U.S. Provisional Patent Application No. 62/933,583, if the typical AR coating were to be applied to a window such as detector window 112, the spherical wave front 152 would propagate through the window without generating any reflections. The output from the photodetector, processed by Fast Fourier Transform (FFT), a technique based on an algorithm that, as known in the art, computes the discrete Fourier transform (DFT) of a sequence, or its inverse (IDFT), would appear as a magnitude versus frequency plot not expected to reveal an observable peak.

In contrast, with a detector window 112 that is free of the AR coating, the spherical wave front gets “trapped’ inside the window and the magnitude versus frequency plot, obtained by FFT, is expected to display a distinct (sharp) peak. While plots can be presented in the frequency domain, FFT can be used to convert the DFT in the frequency domain to a space domain. Such a conversion is expected to produce a distinct (sharp) peak corresponding exactly to the thickness of the window.

In an arrangement utilizing a non-AR coated window, e.g., a non-AR detector window 112, and no live cells, the wave front would not encounter any deflections and simply pass through. As illustrated in FIG. 7, however, when live cells 160 are present, some of the light will become scattered or reflected due to differences in the index of refraction of the live cells relative to that of the culture medium. This effect is expected to reduce the contrast of the etalon window 112 and thus the signal obtained by the FFT. In more detail, the FFT conversion to a space domain is expected to reveal a peak that is smaller in the presence of viable cells when compared to the peak observed in the absence of cells. In short, the scattering produced by the viable cells would be exhibited in a lowering of the contrast of the window etalon.

In practice, live cells 160 in the bioreactor culture medium are expected to change the FFT signal relative to a matched culture medium that is free of live cells. In addition, the magnitude of the peak observed with respect to the length scale will reflect changes in cell viability. For example, a peak will increase as some cells undergo autolysis, since those cells are expected to approach the index of refraction of the culture medium and fewer cell will be encountered by the spherical wave front.

In the embodiment shown in FIG. 7, wave front 152, generated by the lens 126 (held by lens holder 126H), encounters laser window 114 prior to traversing the sample detection area 110, which, as described above, can contain live cells 160. Employing a window 114 that is free of the AR coating and thus is configured as an etalon (as illustrated in FIG. 7).

In some arrangements, the period of the FP etalon is much higher than spectral features of the absorption spectroscopy that may be observed, allowing simultaneous measurements of peaks generated by the etalon described above, in conjunction with optical absorption spectrometry, without deleterious effects. In this respect, one factor to be considered is the thickness of the window employed. A very thin window, for example, might pose problems if the absorption spectral features are comparable to the spectral features of the Fabry Perot etalon.

In many embodiments of the invention, a high frequency signal generated in an etalon such as described above is superimposed onto a single beam transmission signal, obtained, for instance, in the near infrared (NIR) region of the electromagnetic spectrum, namely the region from 780 nanometer (nm) to 2500 nm.

Configurations such as described above also can be employed in multiplexed experiments, where the rocking plate (e.g., rocking plate 14 in FIGS. 1A and 1B) can support two or more flexible bags, each bag having its own in-situ probe, for example. With cell cultures or fermentation processes, multiplexed experiments, using, for instance micro- or mini-bioreactors, make possible the evaluation of process conditions, cell lines, or other variables in an efficient manner and may find applications in scale-up work.

While this invention has been particularly shown and described with references to preferred embodiments thereof, it will be understood by those skilled in the art that various changes in form and details may be made therein without departing from the scope of the invention encompassed by the appended claims.

What is claimed is:

1. An in situ probe, comprising:
a disposable component that is attached to a bioreactor bag.

2. The probe as claimed in claim 1, wherein the disposable component is fused to the bioreactor bag.

3. The probe as claimed in claim 1, wherein the disposable component includes a flange.

4. The probe as claimed in claim 3, wherein the flange is annular.

5. The probe as claimed in claim 1, further comprising a bottom piece that mates with the disposable component.

6. The probe as claimed in claim 5, wherein the bottom piece is also disposable.

7. The probe as claimed in claim 1, further comprising a radiation source and a detector for detecting radiation generated by the radiation source.

8. The probe as claimed in claim 7, wherein the radiation source is contained in a bottom piece that mates with the disposable component.

9. The probe as claimed in claim 8, wherein the detector is contained in a disposable component.

10. The probe as claimed in claim 7, wherein the radiation source is contained in the disposable component.

11. The probe as claimed in claim 10, wherein the detector is contained in a bottom piece that mates with the disposable component.

12. The probe as claimed in claim 1, further comprising a controller for controlling the probe to resolve the spectral response of contents of the bioreactor bag.

13. A rocking bioreactor, comprising:
a bioreactor bag; and
an in situ probe attached to the bioreactor bag.

14. A process for monitoring the rocking bioreactor as described in claim 13.
